(19) United States
(12) Patent Application Publication
(10) Pub. No.: US 2015/0328165 A1
(43) Pub. Date: Nov. 19, 2015

(54) PHARMACEUTICALLY ACCEPTABLE DOSAGE FORM COMPRISING RELEASE OF MULTIPLE DRUGS FROM SINGLE ORAL DOSAGE FORM

(71) Applicant: FTF Pharma Private Limited, Ahmedabad (IN)

(72) Inventors: Jayanta Kumar MANDAL, Ahmedabad (IN); Deepak MAHESHWARI, Ajmer (Raj) (IN)

(21) Appl. No.: 14/713,139

(22) Filed: May 15, 2015

(30) Foreign Application Priority Data
May 16, 2014 (IN) 1664/MUM/2014

(51) Int. Cl. A61K 9/48; A61K 9/20; A61K 31/194; A61K 31/65; A61K 31/4164

(52) U.S. Cl. CPC A61K 9/4808; A61K 31/65; A61K 31/4164; A61K 31/194; A61K 9/2095; A61K 9/4833

(57) ABSTRACT

Present invention relates to a pharmaceutically acceptable dosage form covering multiple drug in one capsule wherein one of these drugs is tetracycline. In the present invention current dosage form tetracycline is in tablet form filled in the capsule. Additionally this dosage may also comprise multiple release of drug.

### FIELD OF INVENTION

The present invention relates to a pharmaceutically acceptable dosage form covering multiple drug release. This dosage form is a capsule which comprises three drugs wherein one of these drugs is tetracycline. In the present invention current dosage form tetracycline is in tablet form filled in the capsule. Additionally this dosage may also comprise multiple release of drug.

### BACKGROUND AND PRIOR ART

To prevent or reduce drug resistance there can be more than one drug are applied simultaneously by separate or combine administration. Many dosage forms are prepared using either bilayer tablet or tablet in capsule or capsule in capsule or liquid filled in capsule or capsule coated with drug etc.

Few of patents listed here. U.S. Pat. No. 7,445,795 discloses inner capsule is filled with one and outer capsule is filled with another drug in the form of liquid.

U.S. Pat. No. 5,310,555 discloses outer capsule contains nutritional supplement and inner capsule contains microorganisms.

U.S. Pat. No. 5,196,205 discloses three drug administration to provide a capsule containing an effective amount of a pharmaceutically acceptable bismuth compound together with enteric coated micro-spherules of an antibiotic of the tetracycline class or penicillin class which capsule also contains an effective amount of a second antibiotic selected from the metronidazole class which second antibiotic is optionally provided in enteric coated micro-spherule form.

U.S. Pat. No. 5,672,359 discloses the capsule comprising three drugs wherein slow and intermediate release drugs are in pellets or granules forms filled in capsule and the outer layer of capsule is coated with immediate release drug.

U.S. Pat. No. 6,350,468 discloses pharmaceutical dosage form and method for carrying out a triple therapy against the microorganisms Helicobacter pylori in a mammal comprising the oral administration to said mammal of a pharmaceutical dosage form comprising an internal capsule placed inside an external capsule, wherein the external capsule comprises a soluble salt of bismuth and a first antibiotic, and the internal capsule comprises a second antibiotic in which the first antibiotic is selected from the group consisting of the nitroimidazoles; and the second antibiotic is selected from the group consisting of the macrolides and the compounds of the family of tetracyclines.

In above mentioned patents three drugs are administered via either capsule in capsule or capsule is coated outer side with drug. In dosage of capsule in capsule it is inconvenient as first to prepare capsule of inside which is to be filled in another capsule. Whereas in dosage of capsule coated outer side with drug, dose reduction may happen either by hurt of drug chemistry or by way of administration or maintenance.

To come out of all said problems the present invention is carried out by filling tablet of tetracycline and granules of at least two drugs in capsule providing multiple drugs in single administration.

### OBJECT OF THE INVENTION

Primary object of the present invention is to provide more than two drugs in single dosage form wherein one drug is tetracycline.

Another object of the present invention is to provide more than two drugs in single dosage form and avoid chemical interaction between bismuth and tetracycline.

It is yet another object of the present invention is to provide combination of more than two drugs either immediate and/or modified release in single dosage form.

The present invention relates to a pharmaceutically acceptable dosage form for multiple drug release. This dosage form is a capsule which comprises three drugs wherein one drug is in tablet form, while second drug and bismuth salt are in blend form. One aspect of the present invention is the use of a pharmaceutical dosage form with multiple drugs to prevent or reduce drug resistance.

### DETAIL DESCRIPTION OF THE INVENTION

Present invention relates to a pharmaceutically acceptable dosage form covering multiple drug release wherein the dosage form is a capsule which comprises three drugs wherein one of these drugs is tetracycline. Tetracycline is in tablet form filled in the capsule. The dosage form further comprises multiple release of drug.

Tetracycline in mini-tablet form incorporated in capsule contains tetracycline or its pharmaceutically acceptable salt and one or more pharmaceutically acceptable excipient amount of tetracycline is in the range of 25-30% w/w.

Further tetracycline can be replace by other antibiotics as an active ingredient preferably selected from the group but not limited to chlortetracycline, doxycycline, glycocycline, guamecycline, lymecycline, methacycline and sancycline.

In the second embodiment of the present invention two more drugs are filled in the capsule in the form of blend. These drugs can be bismuth or its pharmaceutically acceptable salt and metronidazole and its pharmaceutically acceptable salt.

The bismuth salt is preferably selected from the group but not limited to bismuth subcitrate, bismuth aluminate, bismuth carbonate, bismuth citrate, colloidal bismuth subnitrate, bismuth germinate, bismuth germanium oxide, bismuth nitrate, bismuth oxide, bismuth oxychloride, bismuth phosphate, bismuth salicylate, bismuth subcarbonate, bismuth subnitrate, bismuth subsalicylate, bismuth tribromophenate, bismuth trioxyde, bismuth vanadate, and bismuth vanadium tetraoxide between the range of 25-30% w/w. Bismuth salts can also be used in a complex form. For example, bismuth biscalcitrate is a complex form of bismuth subcitrate.

The metronidazole is between the ranges of 25-30% w/w. Metronidazole can be replaced by the active ingredient selected from the group but not limited to apronidazole, azomycine, benzonidazole, carindazole, demetridazole, etanidazole, flunidazole, inisonidazole, nimorazole, ornidazole, panidazole, ronidazole, and tinidazole.

The dosage form of present invention pharmaceutically acceptable excepients can be selected from disintigrant, diluent, lubricant or glidant, coating material etc.

Diluents selected for mini-tablet preparation are selected from the group but not limited to lactose anhydrous, lactose spray dried, directly compressible starch, hydrolysed starch, micro crystalline cellulose (MCC), other cellulose derivative, mannitol, sorbitol, sucrose and dextrose in the concentration range of 1-10% w/w.

Disintegrants used for the preparation of mini-tablet are selected from the group but not limited to starch derivative, clay, cellulose, alginate, PVP, cross linked sodium carboxymethyl cellulose and croscarmellose sodium in the range of 0.5-5% w/w.

Tablet further comprise lubricants selected from the group but not limited to stearic acid, talc, PEG and magnesium stearate within the range of 0.3-15% w/w.

Coating materials used for of mini-tablet are selected from the group but not limited to cellulose acetate phthalate, polyvinyl acetyl phthalate, HPMC, hydroxyl propyl cellulose and opadry white in the range of 0.5-3% w/w.

Blend comprises of diluents selected from the group but not limited to lactose anhydrous, lactose spray dried, directly compressible starch, hydrolised starch, MCC, other cellulose derivative, mannitol, sorbitol, sucrose and dextrose in the concentration range of 1-15% w/w.

Lubricants used for preparation of blend are selected from the group but not limited to stearic acid, talc, PEG and magnesium stearate in the range of 0.3-15% w/w.

Below table represents the composition of present invention.

| No. | Ingredients | Amount % w/w |
|---|---|---|
| 1 | Tetracycline Hydrochloride | 25-30 |
| 2 | Lactose anhydrous | 1-10 |
| 3 | Croscarmellose Sodium | 0.5-5 |
| 4 | Mg Stearate Veg | 0.3-1 |
| 5 | Opadry white 03F180011 | 0.5-3 |
| 6 | Metronidazole | 25-30 |
| 7 | Bismuth Subcitrate Potassium | 25-35 |
| 8 | MCC PH101 | 5-10 |
| 9 | Lactose anhydrous | 10-15 |
| 10 | Talc | 2-15 |
| 11 | Aerosil 200 | 2-10 |
| 12 | Mg Stearate Veg | 0.5-5 |

Process for preparation of this dosage form of used in the present invention includes following steps but not limited to:

### Step 1: Preparation of a Tablet

a. mixing of active ingredient with diluents and disintigrant
b. blend for 1 min at 16 rpm
c. prepared blend is mixed with lubricant and compressed using 6.0 mm round punch which leads to formation of tablet
d. resulting tablet is than coated with coating materials.

### Step 2: Preparation of a Blend

a. mixing bismuth salt and metronidazoleor its pharmaceutically acceptable salt
b. blend the mixture and mixed with diluant
c. blend it for 15 minute at 16 rpm
d. this blend is further mixed with lubricants

### Step 3: Preparation of Capsule Dosage Form

a. fill mini-tablet prepared in step 1 and blend prepared in step 2 into capsules.

The invented dosage form provides drug release from the capsule at various intervals after immersion of the capsule into an aqueous medium e.g. release of tetracycline hydrochloride and metronidazole in 5 minutes after immersion in aqueous medium is 88.80% and 78.90% respectively.

### Examples

The present invention can be described by way of example only. It is to be recognized that modifications falling within the scope and spirit of the description or claims, which would be obvious to a person skilled in the art based upon the disclosure herein, are also considered to be included within the scope of this disclosure.

| Composition | Example 1 | Example 2 | Example 3 | Example 4 |
|---|---|---|---|---|
| Tetracycline | 125 | 125 | 125 | 125 |
| Hydrochloride | | | | |
| Lactose anhydrous | 17.475 | 14.48 | 11.3 | 11.3 |
| Croscarmellose Sodium | 6.25 | 6.25 | 7.25 | 7.25 |
| Mg Stearate Veg | 1.875 | 1.87 | 1.45 | 1.45 |
| Core tablet Wt | 155 | 152 | 145 | 145 |
| Opadry White | 4.65 | 4.5 | 3.5 | 4.35 |
| 03F180011 | | | | |
| Coated Tablet wt | 159.65 | 156.5 | 148.5 | 149.35 |
| Metronidazole | 125 | 125 | 125 | 125 |
| Bismuth Subcitrate | 140 | 140 | 140 | 140 |
| Potassium | | | | |
| MCC PH101 | — | 36 | — | — |
| Lactose anhydrous | — | 21 | 48 | — |
| Talc | — | — | 12 | 12 |
| Aerosil 200 | 4.2 | 4.5 | 4 | 5 |
| Mg Stearate Veg | 2.8 | 3.5 | 4 | 5 |
| Capsule Filled Wt | 272 | 330 | 333 | 287 |
| Total Wt. (Tablet + | 431.65 | 486.5 | 481.50 | 436.35 |
| Blend) | | | | |

Dissolution profiles for the prepared capsules are determined in various media using the following conditions:

| | | | Time | | | | |
|---|---|---|---|---|---|---|---|
| Example | Ingredient | Media | 5 min | 15 min | 20 min | 30 min | 45 min |
| 1 | Bismuth Sub | Water/ | 58.10% | 97.10% | 99.60% | 102.10% | 106.10% |
| | citrate | Paddle with | (10.1) | (3.6) | (3.5) | (3.1) | (2.3) |
| | Potassium | Sinker/ | | | | | |
| | | RPM 75/ | | | | | |
| | | 900 ml/SP - | | | | | |
| | | 5, 15, 20, 30 | | | | | |
| | | & 45 min | | | | | |
| | | (OGD) | | | | | |
| | Metronidazole | 0.1N HCl/ | 88.80% | 95.50% | 96.40% | 97.20% | 97.30% |
| | | 75 RPM/ | (5.3) | (6.1) | (5.8) | (5.1) | (4.9) |
| | | Paddle | | | | | |
| | | with Sinker/ | | | | | |
| | | Vol. - 900 ml | | | | | |
| | | (OGD) | | | | | |
| | Tetracycline | 0.1N HCl/ | 78.90% | 98.20% | 98.40% | 98.20% | 97.50% |
| | Hydrochloride | 75 RPM/ | (16.4) | (1.0) | (0.9) | (0.9) | (0.9) |
| | | Paddle | | | | | |
| | | with Sinker/ | | | | | |
| | | Vol. - 900 ml | | | | | |
| | | (OGD) | | | | | |
| 2 | Bismuth Sub | Water/ | 59.60% | 93.30% | 93.00% | 92.20% | 94.40% |
| | citrate | Paddle with | (11.2) | (1.9) | (2.3) | (2.3) | (2.7) |
| | Potassium | Sinker/ | | | | | |
| | | RPM 75/ | | | | | |
| | | 900 ml/SP - | | | | | |
| | | 5, 15, 20, 30 | | | | | |
| | | & 45 min | | | | | |
| | | (OGD) | | | | | |
| | Metronidazole | 0.1N HCl/ | 91.30% | 95.40% | 95.90% | 95.70% | 95.90% |
| | | 75 RPM/ | (6.1) | (5.3) | (4.6) | (5.0) | (4.6) |
| | | Paddle | | | | | |
| | | with Sinker/ | | | | | |
| | | Vol. - 900 ml | | | | | |
| | | (OGD) | | | | | |
| | Tetracycline | 0.1N HCl/ | 47.30% | 96.30% | 97.40% | 97.0% | 96.30% |
| | Hydrochloride | 75 RPM/ | (48.9) | (4.8) | (4.0) | (4.3) | (3.8) |
| | | Paddle | | | | | |
| | | with Sinker/ | | | | | |
| | | Vol. - 900 ml | | | | | |
| | | (OGD) | | | | | |
| 3 | Bismuth Sub | Water/ | 57.85% | 95.00% | 99.20% | 100.40% | 101.90% |
| | citrate | Paddle with | (6.25) | (4.9) | (2.5) | (1.3) | (0.6) |
| | Potassium | Sinker/ | | | | | |
| | | RPM 75/ | | | | | |
| | | 900 ml/SP - | | | | | |
| | | 5, 15, 20, 30 | | | | | |
| | | & 45 min | | | | | |
| | | (OGD) | | | | | |
| | Metronidazole | 0.1N HCl/ | 85.10% | 96.80% | 96.70% | 97.00% | 97.00% |
| | | 75 RPM/ | (10.5) | (2.1) | (2.2) | (2.1) | (2.2) |
| | | Paddle | | | | | |
| | | with Sinker/ | | | | | |
| | | Vol. - 900 ml | | | | | |
| | | (OGD) | | | | | |
| | Tetracycline | 0.1N HCl/ | 62.5% | 104.5% | 104.10% | 104.20% | 103.50% |
| | Hydrochloride | 75 RPM/ | (51.6) | (0.8) | (0.6) | (1.0) | (0.6) |
| | | Paddle | | | | | |
| | | with Sinker/ | | | | | |
| | | Vol. - 900 ml | | | | | |
| | | (OGD) | | | | | |
| 4 | Bismuth Sub | Water/ | 66.50% | 86.30% | 88.40% | 94.90% | 101.0% |
| | citrate | Paddle with | (9.5) | (1.0) | (2.9) | (2.4) | (2.5) |
| | Potassium | Sinker/ | | | | | |
| | | RPM 75/ | | | | | |
| | | 900 ml/SP - | | | | | |
| | | 5, 15, 20, 30 | | | | | |
| | | & 45 min | | | | | |
| | | (OGD) | | | | | |
| | Metronidazole | 0.1N HCl/ | 84.60% | 96.30% | 97.10% | 97.50% | 98.80% |
| | | 75 RPM/ | (11.0) | (4.0) | (3.4) | (3.5) | (2.0) |
| | | Paddle | | | | | |
| | | with Sinker/ | | | | | |
| | | Vol. - 900 ml | | | | | |
| | | (OGD) | | | | | |
| | Tetracycline | 0.1N HCl/ | 31.30% | 99.60% | 100.30% | 100.20% | 100.50% |
| | Hydrochloride | 75 RPM/ | (58.9) | (5.0) | (3.7) | (3.6) | (0.8) |
| | | Paddle | | | | | |
| | | with Sinker/ | | | | | |
| | | Vol. - 900 ml | | | | | |
| | | (OGD) | | | | | |

1. A dosage form containing more than two drugs in single dosage form wherein one drug is tetracycline or its pharmaceutically acceptable salt.

2. A dosage form containing more than two drugs in single dosage form wherein one drug is metronidazole or its pharmaceutically acceptable salt.

3. A dosage form containing more than two drugs in single dosage form wherein one drug is bismuth citrate or its pharmaceutically acceptable salt

4. A dosage form as claimed in claim 1 wherein the dosage form is in the form of a capsule containing combination of mini-tablet and blend mixture.

5. A dosage form as claimed in claim 1 wherein the dosage form comprises pharmaceutically acceptable excipients like disintigrant, diluent, lubricant and glidant, coating material.

6. A dosage form as claimed in claim 1 wherein tetracycline or its pharmaceutically acceptable salt is in the range of 25-30% w/w.

7. A dosage form as claimed in claim 1 wherein metronidazole or its pharmaceutically acceptable salt is in the range of 25-30% w/w.

8. A dosage form as claimed in claim 1 wherein bismuth or its pharmaceutically acceptable salt is in the range of 25-30% w/w.

9. A dosage form as claimed in claim 5, wherein diluent is between 1-10% w/w, disintigrant is between 0.5-5% w/w, lubricant is between 0.3-15% w/w, coating material is between 0.5-3% w/w.

10. Process for preparation of a dosage form containing more than two drugs in single dosage form comprises
Step 1: preparation of a tablet
a. mixing of tetracycline or its pharmaceutically acceptable salt with diluents and disintigrant
b. blend the mixture
c. prepared blend is mixed with lubricant and compressed to tablet
d. resulting tablet is than coated with coating materials.
Step 2: preparation of a blend
a. mixing bismuth salt and metronidazoleor its pharmaceutically acceptable salt
b. blend the mixture and mixed with diluent
c. blend it
d. this blend is further mixed with lubricants
Step 3: preparation of capsule dosage form
a. fill the tablet prepared in step 1 and blend prepared in step 2 into capsules.

11. The dosage form according to claim 2, wherein the dosage form is in the form of a capsule comprising a combination of mini-tablet and blend mixture.

12. The dosage form according to claim 3, wherein the dosage form is in the form of a capsule comprising a combination of mini-tablet and blend mixture.

13. The dosage form according to claim 2, wherein the dosage form comprises one or more pharmaceutically acceptable excipients selected from the group consisting of disintigrant, diluent, lubricant and glidant, and coating material.

14. A dosage form according to claim 3, wherein the dosage form comprises one or more pharmaceutically acceptable excipients selected from the group consisting of disintigrant, diluent, lubricant and glidant, and coating material.

15. The dosage form according to claim 13, wherein the diluent is between 1-10% w/w, the disintigrant is between 0.5-5% w/w, the lubricant is between 0.3-15% w/w, and the coating material is between 0.5-3% w/w.

16. The dosage form according to claim 14, wherein the diluent is between 1-10% w/w, the disintigrant is between 0.5-5% w/w, the lubricant is between 0.3-15% w/w, and the coating material is between 0.5-3% w/w.
